(19) United States
(12) Patent Application Publication
(10) Pub. No.: US 2026/0336889 A1
(43) Pub. Date: Oct. 29, 2026

(54) ROBOTIC ENCODING SYSTEM AND METHOD

(71) Applicant: Caring Closure International LLC, Greenwood Village, CO (US)

(72) Inventors: Milton Cohen, Greenwood Village, CO (US); Keir Hart, Cleveland, CO (US)

(21) Appl. No.: 19/111,466

(22) PCT Filed: Sep. 14, 2023
(86) PCT No.: PCT/US2023/032699

§ 371 Date: Mar. 13, 2025

Related U.S. Application Data
(60) Provisional application No. 63/406,549, filed on Sep. 14, 2022.

(51) Int. Cl. G07G 1/00

(52) U.S. Cl. CPC G07G 1/0018

(57) ABSTRACT

A lockable container encoding system and method is disclosed. The lockable container encoding system is for use in the manufacture of encodable lockable containers, wherein the encodable lockable container comprises an annular sidewall having a plurality of annular rings, the annular rings having an inner and outer surface, the inner surface featuring a plurality of selectable removable tabs for the encoding of lockable containers. The lockable container encoding system includes a tumbler for receiving unencoded encodable lockable containers, a receptor having a sidewall dimensioned and configured to hold a plurality of encodable lockable containers, a first articulating arm comprising a plurality of ring manipulators, a vision system in communication with the ring manipulators to monitor the physical position of the plurality of annular rings of the encodable lockable container, and a second articulating arm comprising a plunger having a shearing blade; wherein the shearing blade comprises guide rails couplable to the shearing blade, wherein the shearing blade removes a plurality of selected removable tabs from an unencoded lockable container to encode the container.

### CROSS REFERENCES TO RELATED APPLICATIONS

The following application claims priority under 35 U.S. C. § 119(e) to co-pending U.S. Provisional Patent Application Ser. No. 63/406,549 filed Sep. 14, 2022 entitled ROBOTIC ENCODING SYSTEM AND METHOD. The above-identified application is incorporated herein by reference in its entirety for all purposes.

### TECHNICAL FIELD

The present disclosure generally relates to a robotic encoding system for a lockable container and method of operating same, and more particularly, a robot encoder that encodes the cap of a lockable container.

### BACKGROUND

Tamper-proof or child-resistant containers, such as those used, for example, for distributing pharmaceutical medications, are widely used to restrict access to the contents. To provide greater security, especially for controlled substances, locking caps with selectively-programmable rings may be used. For example, U.S. Pat. No. 8,875,915 (“Container having a Programmable Combination Locking Cap”) and U.S. Pat. No. 10,842,713 (“Lockable Container”), both issued to Secure Medication System, LLC of Fort Collins, CO. and are incorporated in their entirety herein by reference. The above patents describe a number of embodiments of containers that include individually programmable combination locking caps. In some embodiments, the locking cap includes a number of rotatable rings that may be aligned adjacent to one another on the cap.

According to some embodiments of the '915 and '713 patents, tabs formed on the inner surface of these rings operate in conjunction with projections formed on a canister portion of the container to allow/prevent the cap from being removed from the canister portion. Access to the contents contained in the canister portion is made possible, however, by removing one tab on each ring, so that there is nothing to interfere with the projections when the plural rings are positioned so that the removed tabs are aligned along a vertical axis associated with the projections. To facilitate aligning the rings to unlock the cap and canister portion, alphanumerical indicia may be formed on the outer surface of the ring. The indicia that position the removed tabs along the vertical axis associated with the projections on the canister portion correspond to the combination code for unlocking the container. In some implementations, the consumer may create her own combination code; in other implementations, the pharmacist may create the combination code.

In some implementations (e.g., containers for medications), the corresponding industry and customary sizes that consumers are familiar with may dictate the size of future canister and cap combinations. As a result, whereas a combination code having three or more digits (on three or more rings) may be more secure than a combination code having just two digits (on two rings), because the size of the container and the portion of that overall size that may be available for the rotatable rings may be standardized, having more rings may mean that the rings are less wide than solutions with fewer rings, Problematically, some consumers may find it more difficult to see the alphanumerical indicia on the less wide rings than on wider rings. These same and other consumers may also find it more difficult to manipulate the less wide rings than the wider rings. Furthermore, rotating three or more rings requires aligning a combination code that includes an equal number of alphanumerical indicia. Disadvantageously, rotating one ring may affect the alignment of adjacent rings (e.g., due to “sticky” rings), requiring the process to take longer and the rings having to be moved back and forth to provide an exact alignment.

Tolerances in manufacturing of, for example, the caps, the rotatable rings, the tabs on the rings, the flanges, and the canister portion, as well as the number of rotatable rings, may make unlocking the cap and canister combination, as well as opening the container, more difficult. This phenomenon may be referred to as ring indexing tolerance. Ring indexing tolerance may manifest among senior consumers whose eyesight or digital dexterity may not permit or may prolong a perfect alignment of the combination code. Alternatively, or in addition, the relative alignment of the alphanumerical indicia on outer surface of the ring with the tabs on the inner surface of the ring during manufacture may be off sufficiently, such that, even if combination code is properly aligned along the vertical axis of the projections formed on the canister portion, one or more tabs may interfere with the projections, preventing removal of the cap from the canister portion.

Finally, single-piece caps for containers often include, for holding the rings, a cylindrical member that is slightly tapered. In some instances, the tapering may induce some free play in the rings, affecting the ability to exactly align the appropriate combination code.

### SUMMARY

Robotic encoding of a lockable container and a method are described. A lockable container encoding system for use in the manufacture of lockable containers, wherein said lockable container comprises a plurality of annular rings, the annular rings having an inner and outer surface, the inner surface comprising a plurality of selectably removable tabs for the encoding of lockable containers. The lockable container encoding system comprising a processing device having a processor configured to perform a predefined set of operations in response to receiving a corresponding input from an external source, a robot encoder in communication with the computer and the processing device, the processing device for instructing the robot encoder to encode a particular personal identification number (PIN) into the selectably removable tabs. The robot encoder housing a plurality of ring manipulators to rotate the plurality of annular rings, a means of monitoring a physical position of the plurality of annular rings of the encodable lockable container, and a tab removal device to remove a plurality of selected removable tabs from an unencoded lockable container to encode said container, wherein responsive to instructions from one of the processing device or the point of sale computer, the robot encoder encodes the unencoded lockable container to encode said container.

Another aspect of the present disclose is a non-transitory computer readable medium storing instructions executable by an associated processor to perform a method for encoding lockable containers, comprising receiving a refill instruction and an associated personal identification number (PIN), intaking a lockable container comprising a plurality of annular rings, the annular rings having an inner and outer surface, the inner surface comprising a plurality of selectably removable tabs for the encoding of lockable containers. aligning the annular rings to selectively remove the tabs to create an encoded lockable container conforming to the associated PIN, removing the tabs to conform to the associated PIN, and outputting an encoded lockable container.

Yet another aspect of the present disclose is a method for encoding a lockable containers, wherein said lockable container comprises a plurality of annular rings, the annular rings having an inner and outer surface, the inner surface comprising a plurality of selectably removable tabs for the encoding of lockable containers. The method comprising intaking an instruction and an associated personal identification number (PIN), aligning the annular rings to selectively remove the tabs to create an encoded lockable container conforming to the associated PIN, removing the tabs to conform to the associated PIN, and outputting an encoded lockable container.

### BRIEF DESCRIPTION OF THE DRAWINGS

The foregoing and other features and advantages of the present disclosure will become apparent to one skilled in the art to which the present disclosure relates upon consideration of the following description of the invention with reference to the accompanying drawings, wherein like reference numerals, unless otherwise described refer to like parts throughout the drawings and in which:

FIG. 1A is a front perspective view of a lockable container that may be created according to one embodiment of the present disclosure;

FIG. 1B is an exploded view of FIG. 1 in accordance with one example embodiment of the present disclosure;

FIG. 2A is a schematic diagram of a robot encoder system, in accordance with one example embodiment of the present disclosure;

FIG. 2B is a schematic diagram depicting a method of operation of a robot encoder in accordance with one example embodiment of the present disclosure;

FIG. 3A is a schematic diagram of a robot encoder in accordance with a second embodiment of the present disclosure;

FIG. 3B is a schematic diagram of a robot encoder in accordance with another example embodiment of the present disclosure;

FIG. 3C is a schematic diagram of a robot encoder in accordance with yet another example embodiment of the present disclosure

FIG. 4 is a schematic diagram of an in-line high velocity robot in accordance with one embodiment of the present disclosure; and

FIG. 5 is an illustration of a robot encoder in accordance with yet another example embodiment of the present disclosure.

### DETAILED DESCRIPTION

Referring now to the figures wherein like numbered features shown therein refer to like elements throughout unless otherwise noted. The present disclosure relates generally to a robotic encoding of a lockable container and method, and more particularly, a robot encoder that encodes the cap of a lockable container.

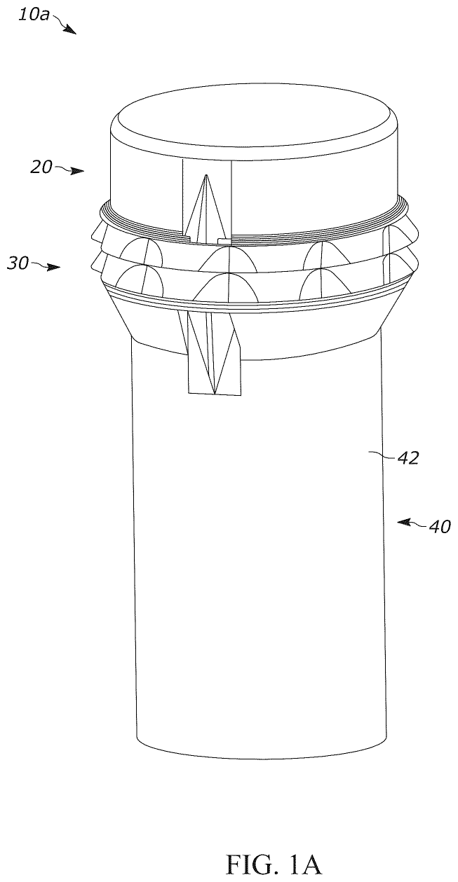

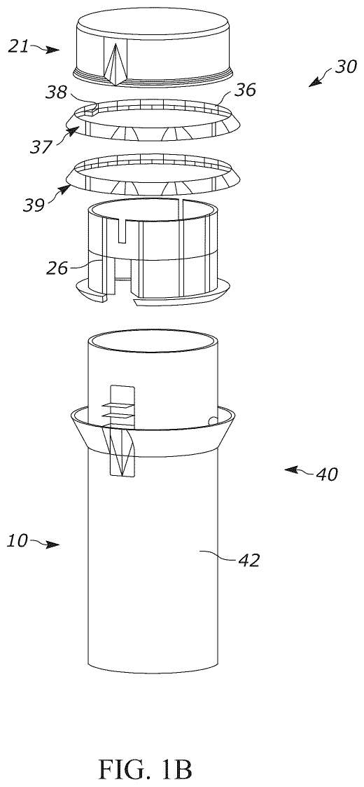

Referring to FIGS. 1A and 1B, respectively, isometric and exploded views of a first embodiment of a lockable programmable container 10 are shown. In some embodiments, the lockable programmable container 10 may include a first element 20, a plurality of rotatable rings 30, and a second element 40. In one example embodiment, the first element 20 includes multiple portions, e.g., a retainer/cap 21 and a cylindrical member 26. Advantageously, for this first embodiment, the multi-piece (e.g., two-piece) first element 20 may be structured and arranged to be used as the cap 21 of the container 10. While any number of rotatable rings 30 may be used, two rings 30 are shown in the figures. For the first embodiment, the rotatable rings 30 are operatively disposed on the first element 20 (cap portion) of the container 10; for the second embodiment, discussed below, the rotatable rings 30 are operatively disposed on the second element 40 or canister portion 42 of the container 10.

As shown in FIG. 1B, the rotatable rings 30 are annular, having an inner surface 36 and an outer surface 37. In some implementations, a plurality of removable tabs 38 may be formed on the inner surface 36 of each ring 30, while an alphanumeric character 39 may be located on the outer surface 37 of each ring 30. The alphanumerical characters 39 enable the consumer, pharmacist, or authorized third party to create a multi-digit (e.g., a two-digit, a three digit, etc.) combination code for the purpose of locking and selectively unlocking the container 10 (by re-entering the appropriate combination code). Preferably, the size and location of each alphanumerical character 39 on the outer surface 37 of the ring 30 corresponds to a discrete removable tab 38 formed on the inner surface 36 of the ring 30. In another example embodiment, the character 39 comprise color coding. In yet another example embodiment, the alphanumerical character 39 is offset or inset. The inner diameter of the ring 30 may be designed so that, once the rings 30 are placed on the cylindrical member 26, the rings 30, and especially the removable tabs 38 on the inner surface 36, will rotate smoothly about the circumference of the cylindrical member 26. Removal of the tabs 38 and creation of a combination code are discussed below.

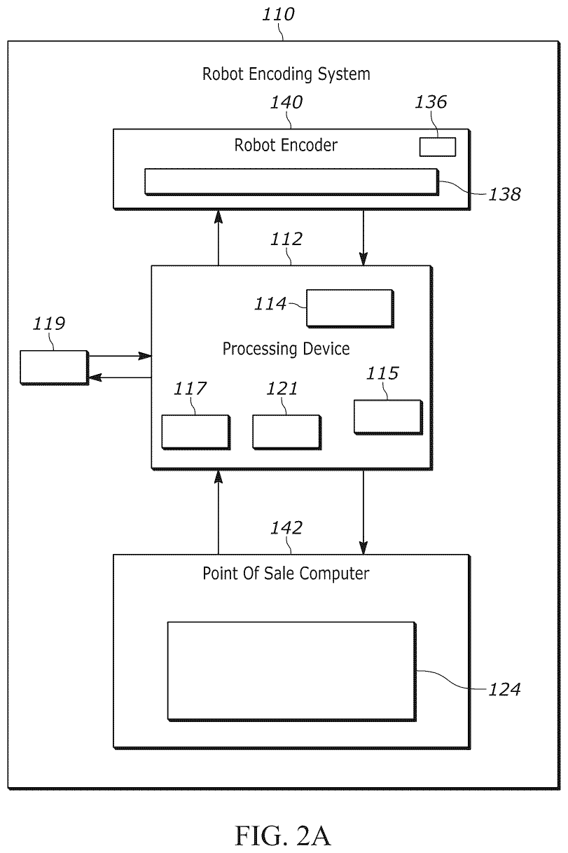

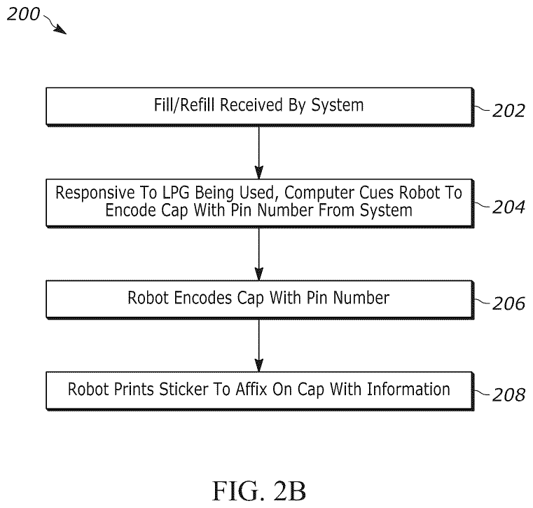

FIG. 2A illustrates a schematic diagram of a robotic encoding system 110, in accordance with one of the exemplary embodiments of the disclosure. In one example embodiment, the robotic encoding system 110 includes a processing device 112. In one example embodiment, the processing device 112 includes a computing device 115 (e.g. a database server, a file server, an application server, a computer, or the like) with computing capability and/or a processor 114. The processor 114 comprises central processing units (CPU), such as a programmable general purpose or special purpose microprocessor, and/or other similar device or a combination thereof.

The processing device 112 would generate outputs based upon inputs received from a robot encoder 140 and/or a point of sale (POS) computer 142, cloud storage, a local input from a user, etc. It would be appreciated by one having ordinary skill in the art that in some embodiments the processing device 112 would include a data storage device 117 in various forms of non-transitory, volatile, and non-volatile memories which would store buffered or permanent data as well as compiled programming codes used to execute functions of the processing device 112. In another example embodiment, the data storage device 117 can be external to and accessible by the processing device 112. In yet another example embodiment, the data storage device 117 includes an external hard drive, cloud storage, and/or other external recording devices 119. The processing device 112 is one of independent of the robot encoder 140 and the point of sale computer 142, or part of one or both of the robot encoder 140 and the point of sale computer 142

In one example embodiment, the processing device 112 comprises one of a remote or local computer system 121. The computer system 121 includes desktop, laptop, tablet hand-held personal computing device, IAN, WAN, WWW, and the like, running on any number of known operating systems and are accessible for communication with remote data storage, such as a cloud, host operating computer, via a world-wide-web or Internet.

In another example embodiment, the processing device 112 comprises a processor, a microprocessor, a data storage, computer system memory that includes random-access-memory (“RAM”), read-only-memory (“ROM”) and/or an input/output interface. The processing device 112 executes instructions by non-transitory computer readable medium either internal or external through the processor that communicates to the processor via input interface and/or electrical communications, such as from the robot encoder 140 and/or the point of sale computer 142. In yet another example embodiment, the processing device 112 communicates with the Internet, a network such as a LAN, WAN, and/or a cloud, input/output devices such as flash drives, remote devices such as a smart phone or tablet, and displays.

In one example embodiment, the point of sale computer 142 includes an interactive display 124. In one example embodiment, the interactive display 124 is for receiving tactile input. In another example embodiment, the point of sale computer 142 includes a secondary device, such as a smart phone, tablet, desktop, laptop, or the like. In another example embodiment, the robot encoder 140 includes an interactive display 138. In one example embodiment, the interactive display 138 is for receiving tactile input and/or displaying information. In another example embodiment, the robot encoder 140 includes printer 136 for printing information. In yet another example embodiment, a printer 136 is part of the robot encoder system 110.

In another example embodiment, the robot encoder 140 includes a secondary device, such as a smart phone, tablet, desktop, laptop, or the like. In another example embodiment, the processing device 112, an SD-card-writer (e.g., for data retrieval), and the interactive display 124 (e.g., a touch-screen status-and-control LCD display) are housed in a separate module connected to the processing device 112 and/or the robot encoder 140 via short range wireless signals, WIFI, and/or corded communication.

In FIG. 2B, a method 200 of robotically encoding the canister 42 or cap 21 of the container 10 featuring a plurality of rings 30 that was shown in FIGS. 1A and 1B is illustrated. In one example embodiment, the robot encoder 140 is coupled to a retail or prescription remote or local POS computer system 142 and/or the processing device 112. At 202, in one example embodiment, a prescription fill or refill is received by the processing device 112 from the POS computer system 142. In the example embodiment, the fill or refill is assigned to a patient from a stored patient directory in the memory of said POS computer system 142 and communicated to the processing device 112. In one example embodiment, the robot encoder 140 features localizer performance with vertical guidance (LPG) remotely coupled to the POS computer system 142. At 204A, (optional) wherein, responsive to the LPG being used for the fill/refill, the POS computer system 142 and/or the processor 112 instructs the robot encoder 140 to encode a lockable container 10 (see FIGS. 1A and 1B) with a patient's personal identification number (PIN)number stored in the computer's 142 PIN field. At 206, the robot encoder 140 encodes the cap 21 or canister 42 with the patient PIN number retrieved from the system 142. At 208, the robot encoder 140 prints a label or sticker with fill or patient identifying information and affix it to the cap 21 or canister 42 (see FIGS. 1A-1B).

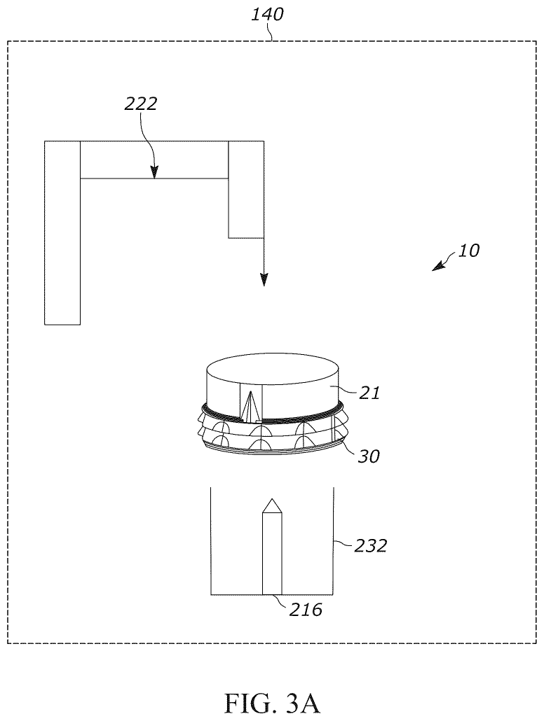

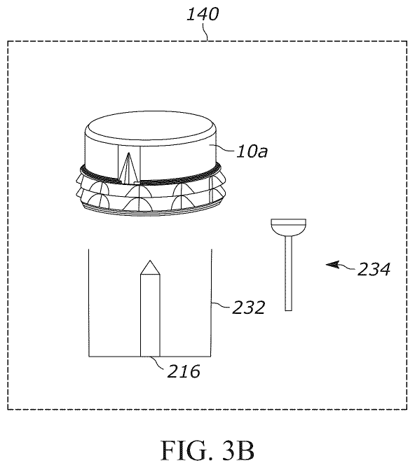

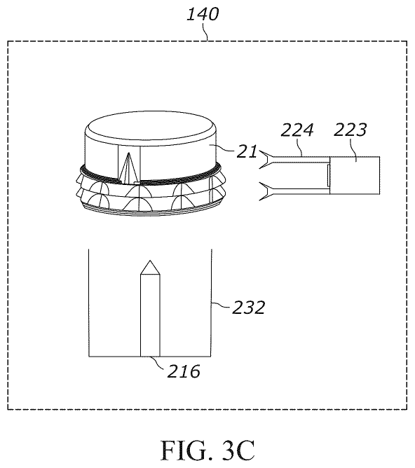

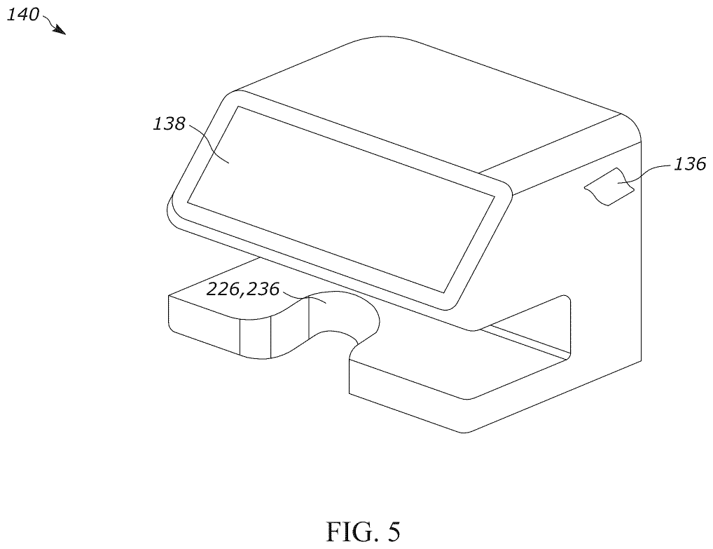

In FIG. 3A, the structure of the robot encoder 140 and method of operation is illustrated. In one example embodiment, an unencoded portion of a lockable container 10 (see FIGS. 1A-1B) either the first or second portion 20, 40, is inserted into an entry area 226, see FIG. 5, of the robot encoder 140. In one example embodiment, the unencoded portion is on the cap portion 21 of the container 10; in another embodiment, the unencoded portion is disposed on the canister portion 42 of the container 10.

In another example embodiment, such as illustrated in FIG. 3A, either the first or second portion 20, 40, (see FIGS. 1A and 1B) is inserted into an entry area 226 of the robot encoder 140 (see FIG. 5). In this example embodiment, the entry area 226 is one of a robotic articulating arm, an XY coordinate machine with vacuum plunger and/or gripper, a delta robot, a feeder bowl, a hopper and/or conveyor that leads to a funneled or chamfered entrance into the robot encoder 140 to orient either the first or second portion 20, 40, respectively.

In this example embodiment, the robot encoder 140 houses an actuator (e.g., an articulating arm 222). The articulating arm 222 selectively stamps the removable tabs 38 on the inner surface 36 of the unencoded container 10 down into a tab removal device 216. In one example embodiment, the tab removal device 216 is one of a shearing blade, and/or a heated blade. In one example embodiment, the tab removal device 216 itself remains static, thus removing selected removable tabs 38 and encoding the patient's PIN number onto the container 10 (e.g., by selectively removing the removable tabs 38 on the inner surface 36).

In one example embodiment, such as illustrated in FIG. 3B, the robot encoder 140 houses a plunger featuring tab removal devices 216 that extend in the direction of the entry area 226 of the robot encoder 140. In this example embodiment, the tab removal device 216 interact with the unencoded portion of the unencoded container 10 to encode the patient's PIN number onto the container 10 (e.g., by selectively removing the removable tabs 38 on the inner surface 36.) In one example embodiment, the robot encoder 140 is aided by a visual system 223 guided ring manipulators 224, which turn/rotate the rings 30 of the unencoded container 10 to configure with a patient PIN number. In one example embodiment, the guided ring manipulators 224 comprises one or more of rollers (e.g., textured or have rubbery ends), cams, and/or gripper actuators. In another example embodiment, the visual system 223 includes video, such as a single or dual camera, and/or a laser. One such example laser is the Keyence laser micrometer. In another example embodiment, the visual system 223 is omitted. In this example embodiment, the guided ring manipulators 224 comprises a nut driver or glove structure that interacts with the alphanumeric characters and/or an offset/inset on the rings to manipulate the one or more rings 30 into an encoding position, to selectively remove the removable tabs 38 to encode the container 10. In one such example embodiment, the guided ring manipulators 224 will interact with a first ring and a second ring of the one or more rings, and turn both rings to the encoding position of the first ring, then the guided ring manipulators 224 will partially retract to interact solely with the second ring, and turn the second ring into the encoding position of the second ring. In this example embodiment, the guided ring manipulators 224 read an inset or an offset that signifies a position of the one or more rings, and rotates the rings accordingly.

The entry area 226 provides a channel 228 for the unencoded containers 10 to enter the robot encoder 140. The entry arca 226 intakes the unencoded containers 10 into a receptor in an orientation configured to match the selected patient PIN number (e.g., via a conveyer, suction, gravity, etc).

In the above embodiment, the tab removal device 216 is used to remove tabs 38 on the unencoded container 10 that do correspond with the selected patient PIN number, The tab removal device 216 comprises guide rails 232 to ensure that the tab removal device 216 will reach the removable tabs 38 of the unencoded container 10. The tab removal device 216 are one of movable or static in different embodiments of the robot encoder 140. Once the tab removal device 216 has removed the removable tabs 38 that do correspond with the patient PIN number, a retracting actuator 234 (e.g., a push arm) pushes the finished container 10a to a finished cap opening 236 formed in the robot encoder 140, where a user may remove the encoded container 10a. In one example embodiment, the finished cap opening 236 and the-entry area 226 are the same area/egress of the robot encoder 140.

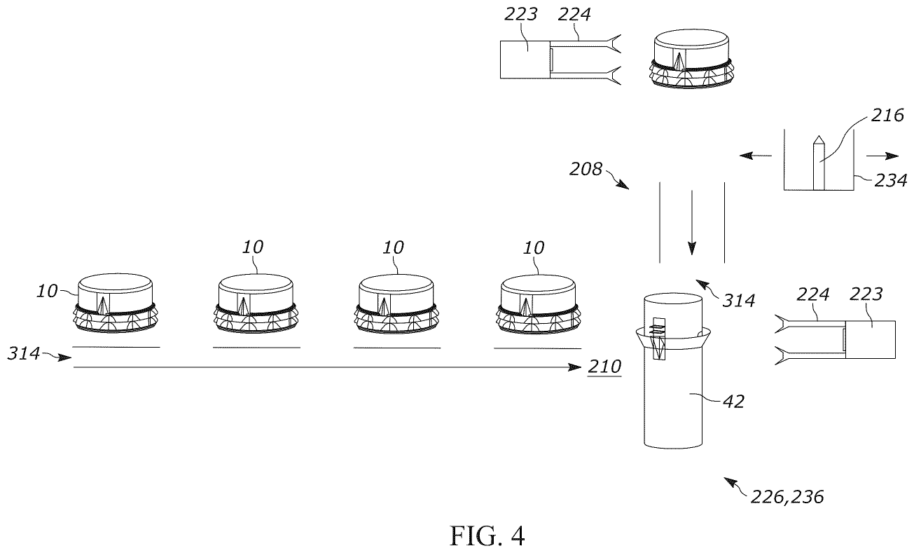

In another example embodiment, such as illustrated in FIG. 4, the robot encoder 140 is an in-line high velocity robot. The in-line high velocity robot encoder 140 comprises a separate capping line achieved by localized performance with vertical guidance (not shown). In one example embodiment, the in-line high velocity robot encoder 140 is a spur on an existing container fill or refill assembly line. In one example embodiment, the in-line high velocity robot encoder 140 includes at least one of the visual system 223 guided aligning arms 224, one or more tab removal devices 216, a conveyor 208, and/or other features. In another example embodiment, the aligning manipulators 224 interact with and inset/offset of the rings 30, to move rings 30 on an unencoded container or cap 10 so that a shearing blade 216 may remove removable tabs 38

Advantageously, visual system 223 guided aligning manipulators 224 move rings 30 on an unencoded container or cap 10 so that a shearing blade 216 may remove removable tabs 38 of the unencoded cap or container 10 that do correspond with a selected patient PIN number. The shearing blade 216 used in this embodiment is retractable. Once encoded, the encodable container 42 or cap 21 drops through a passage 208 defined within the robot encoder 142 and onto a corresponding container 10 travelling along a conveyer 210 to form a finished lockable container 10a (see FIG. 1A). The conveyor 210 features orienting mechanics, such as at least one of visual system 223 guided aligning manipulators 224 to match the cap 21 or container 42, with a corresponding encoded cap or container. In this example embodiment, the visual system 223 guided aligning manipulators 224 lock the container by scrambling the rings 30 of the encodable cap 10. In another example embodiment, the aligning arms 212 scramble the rings 30 of the encodable cap 10 in a separate station (not shown) after the finished lockable container 10a is capped and ready for removal.

In the foregoing specification, specific embodiments have been described. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . ”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. In one non-limiting embodiment the terms are defined to be within for example 10%, in another possible embodiment within 5%, in another possible embodiment within 1%, and in another possible embodiment within 0.5%. The term “coupled” as used herein is defined as connected or in contact either temporarily or permanently, although not necessarily directly and not necessarily mechanically. A device or structure that is “configured” in a certain way is configured in at least that way, but may also be configured in ways that are not listed,

To the extent that the materials for any of the foregoing embodiments or components thereof are not specified, it is to be appreciated that suitable materials would be known by one of ordinary skill in the art for the intended purposes.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

We claim:

1. A lockable container encoding system for use in the manufacture of a lockable container, wherein said lockable container comprises a plurality of annular rings, the annular rings having an inner and outer surface, the inner surface comprising a plurality of selectably removable tabs for the encoding of the lockable container, the lockable container encoding system comprising:
a processing device having a processor configured to perform a predefined set of operations in response to receiving a corresponding input from an external source;
a robot encoder in communication with a computer and the processing device, the processing device for instructing the robot encoder to encode a particular personal identification number (PIN) into the selectably removable tabs;
the robot encoder housing:
a plurality of ring manipulators to rotate the plurality of annular rings;
a means of monitoring a physical position of the plurality of annular rings of the encodable lockable container; and
a tab removal device to remove a plurality of selected removable tabs from an unencoded lockable container to encode said container,
wherein responsive to instructions from one of the processing device or a point of sale computer, the robot encoder encodes the unencoded lockable container to encode said container.

2. The lockable container encoding system of claim 1 wherein the means of monitoring a physical position comprises a vision system in communication with the ring manipulators to monitor.

3. The lockable container encoding system of claim 1, wherein the tab removal device comprises a shearing blade.

4. The lockable container encoding system of claim 3, wherein the shearing blade is one of retractable or static.

5. The lockable container encoding system of claim 1, further wherein, the robot encoder houses an entry area having a sidewall dimensioned and configured to intake and hold a plurality of encodable lockable containers.

6. The lockable container encoding system of claim 1, further wherein the plurality of ring manipulators comprise a first articulating arm.

7. The lockable container encoding system of claim 1, further comprising an articulating arm comprising a plunger, said plunger for causing interaction between the tab removal device and the plurality of selected removable tabs.

8. The lockable container encoding system of claim 1, wherein the external source is a point of sale computer.

9. A non-transitory computer readable medium storing instructions executable by an associated processor to perform a method for encoding a lockable container, comprising:
receiving a fill or refill instruction and an associated personal identification number (PIN);
intaking a portion of a lockable container comprising a plurality of annular rings, the annular rings having an inner and outer surface, the inner surface comprising a plurality of selectably removable tabs for the encoding of lockable containers;
aligning the annular rings to selectively remove the tabs to create an encoded lockable container conforming to the associated PIN;
removing the tabs to conform to the associated PIN; and
outputting at least a portion of an encoded lockable container.

10. The method of claim 9, the receiving the fill or refill instruction comprising receiving refill instructions from a point of sale computer.

11. The method of claim 9, the intaking the portion of the lockable container comprising intaking a lockable container into an encoding robot.

12. The method of claim 9, the aligning the annular rings comprising aligning the annular rings utilizing a vision system and an articulating robot housed within an encoding robot.

13. The method of claim 9, further comprising outputting a printout of a prescription label.

14. The method of claim 9, further comprising rotating the annular rings utilizing a plurality of ring manipulators to align the annular rings.

15. The method of claim 14, further comprising the plurality of ring manipulators communicating with a vision system to monitor the physical position of the plurality of annular rings of the encodable lockable container.

16. The method of claim 9, further comprising removing the tabs utilizing a tab removal device.

17. The method of claim 9, further comprising removing the tabs utilizing a shearing blade.

18. A method for encoding a portion of lockable containers, wherein said portion of a lockable container comprises a plurality of annular rings, the annular rings having an inner and outer surface, the inner surface comprising a plurality of selectably removable tabs for the encoding of lockable containers, the method comprising:
intaking a fill or refill instruction and an associated personal identification number (PIN);
aligning the annular rings to selectively remove the tabs to create at least a portion of an encoded lockable container conforming to the associated PIN;
removing the tabs to conform to the associated PIN; and
outputting the at least a portion of the encoded lockable container.

19. The method of claim 18, the aligning the annular rings comprising aligning the annular rings utilizing a vision system and an articulating robot housed within an encoding robot.

20. The method of claim 18, further comprising rotating the annular rings utilizing a plurality of ring manipulators to align the annular rings.
